(19) United States
(12) Patent Application Publication
(10) Pub. No.: US 2026/0256745 A1
(43) Pub. Date: Sep. 3, 2026

(54) Fenbendazole to cure prevent and or treat Diabetes

(71) Applicant: Linda J. Neal, Broken Arrow, OK (US)

(72) Inventor: Linda J. Neal, Broken Arrow, OK (US)

(21) Appl. No.: 19/414,580

(22) Filed: Dec. 10, 2025

(51) Int. Cl. A61K 31/4184; A61K 31/085; A61K 31/12; A61K 31/216; A61K 31/352; A61K 31/4375; A61P 3/10; A61P 25/16

(52) U.S. Cl. CPC A61K 31/4184; A61K 31/085; A61K 31/12; A61K 31/216; A61K 31/352; A61K 31/4375; A61P 3/10; A61P 25/16

(57) ABSTRACT

Fenbendazole offers a cure for Diabetes. The glucose levels in diabetes can be normalized by clearing tau and microtubules, resulting in clearing of Hyperglycemia. Hyperglycemia refers to high blood glucose readings, taken from the blood vessels. The glucose is having difficulty passing into some cells through thick microtubules. As seen in recent High Resolution Microscopy: hundreds or thousands of microtubules can be found in a single problematic cell. “during diabetes, microtubules are much denser inside beta cells.” By clearing excess tau oligomers and microtubules, Fenbendazole normalizes the glucose levels. Parkinson's disease also has Hyperglycemia and excess tau oligomers. “tau aggregation correlates with motor deficits and degeneration of dopamine-producing regions of the brain” in Parkinson's. Retinal manifestations of Tau or Amyloid are a biomarker. From the eyes, Tau has been known to proliferate through nerve cells, reaching the brain.

### BRIEF DESCRIPTION OF DRAWINGS

The patent or application file contains at least one drawing executed in color. Copies of this patent or patent application publication with color drawing(s) will be provided by the Office upon request and payment of the necessary fee.

A portion of the disclosure of this patent document contains material which is subject to (copyright or mask work) protection. The (copyright or mask work) owner has no objection to the facsimile reproduction by anyone of the patent document or the patent disclosure, as it appears in the Patent and Trademark Office patent file or records, but otherwise reserves all (copyright or mask work) rights whatsoever.

“On rare occasions, color drawings may be necessary as the only practical medium by which to disclose the subject matter sought to be patented in a utility patent application.” USPTO

FIG. 1. Fenbendazole cleared metastatic cancer

FIG. 2. Tau, inside neuron cells

FIG. 3. Fenbendazole

FIG. 4. Mebendazole

FIG. 5. Tau binding to tubulin subunits of Microtubules

FIG. 6. Tau, seen as stringers of protein beads

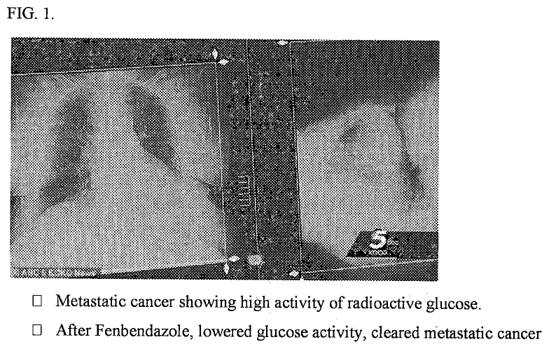

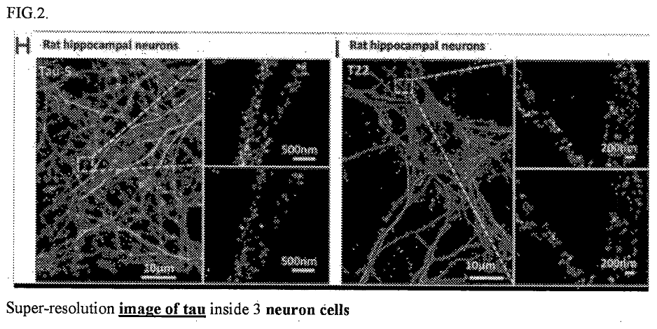

### FIELD OF INVENTION

This invention is related to the area of pharmacology, Diabetes and Parkinson's therapeutics.

### HISTORY Provisional Application 63/811,593 May 24, 2025 01:03:45 PM Z ET

### BACKGROUND OF (THE) INVENTION

Fenbendazole has been used for 50 years to treat parasites. According to the USDA, Fenbendazole “starves” the parasites by destabilizing Microtubules; parasites require and feed on high glucose levels in the blood, known as Hyperglycemia.
[USDA] United States Department of Agriculture May 13, 2020 line 69, 71, 122, 249, 335,709 [USDA] Technical Report—Fenbendazole—May 13, 2020
biologyinsights.com>does-eating-sugar-actually . . . Does Eating Sugar Actually Feed Parasites?—Biology Insights “Jun. 29, 2025—For parasites in the bloodstream, like the trypanosomes that cause sleeping sickness, they feed on glucose circulating in the blood. The direct link between a host's sugar . . .

### EXAMPLE 1 FENBENDAZOLE HAS “STARVED” PARASITES OF GLUCOSE

“Fenbendazole and mebendazole, another benzimidazole, were also tested along with a placebo to treat patients in Udaipur, India who were infected with human pinworm (Enterobius vermicularis) . . . . All patients treated with fenbendazole and mebendazole recovered; the patients receiving the placebo showed no improvement. Minor side effects reported by a few of the test subjects included constipation and a burning sensation during urination (Bhandari and Singhi 1980).”

### Technical Evaluation Report Fenbendazole

May 13, 2020 line 221-244 [USDA]
Technical Report—Fenbendazole—May 13, 2020

### EXAMPLE 2 FENBENDAZOLE HAS “STARVED” CANCER CELLS

Next, it was discovered that Fenbendazole would prevent cancer. Feeding Fenbendazole to laboratory mice cleared the source of the Hyperglycemia, and protected the mice from attempts to induce cancer. Cancer cells require high levels of glucose in the blood.

AI: “Yes, fenbendazole can kill cancer cells through its interaction with microtubules. It acts as a microtubule destabilizing agent, meaning it disrupts the normal structure and function of microtubules. It can affect energy metabolism by disrupting glucose uptake and utilization, and it can induce apoptosis (programmed cell death).

“Of note, pancreatic tau is significantly up-regulated not only in T2DM but also in neurodegenerative diseases like Alzheimer's disease (AD), dementia with Lewy bodies, Parkinson's disease, and incidental Lewy body disease.³ This raises intriguing questions. Is 80 there a common denominator causing tau to be dysfunctional in the whole body?
Li, W., Tiedt, S. & Lo, E. H. Tau in the pancreas: understanding the link between type 2 diabetes mellitus and Alzheimer's disease. Sig Transduct Target Ther 8, 447 (2023). https://doi.org/10.1038/s41392-023-01701-3

### Recognizing Tau

Diabetes and Parkinson's can be defined by an increase in Tau oligomers (Tau, Amyloid, and alpha-synuclein oligomers).

Tau biomarkers include:
Diabetes: Hyperglycemia, erectile dysfunction, death of insulin producing cells.
Parkinson's: Hyperglycemia, erectile dysfunction, death of dopamine producing cells.
   www.medpagetoday.com>neurology>parkinsonsdisease
   Parkinson's Risk Rises With Metabolic Syndrome—Medpage Today
   “Previous studies have shown a relatively consistent association between hyperglycemia and Parkinson's disease, . . .
      parkinsonsnewstoday.com>news>diabetes-bloodDiabetes, Blood Sugar Tied to Parkinson's Nerve Cell Damage . . .
   Apr. 19, 2022 “Diabetes is a chronic condition characterized by hyperglycemia, or high blood sugar levels. The disease has been associated with an increased risk of developing Parkinson's. Further, diabetes and high blood sugar have both been implicated in a faster progression of Parkinson's motor symptoms.
      pmc.ncbi.nlm.nih.gov>articles>PMC11831814
   Parkinson's disease and glucose metabolism impairment—PMC
   “Accumulating evidence from epidemiological studies suggests that hyperglycemia and diabetes may increase the risk of sporadic PD.

### Tau Seeds, Migrates, and Spreads:

Tau in pancreas predicts the death of insulin producing cells. (Diabetes)
Tau in the brain predicts the death of dopamine producing cells. (Parkinson's)

Diabetes is often diagnosed after many of the pancreatic cells have died and the amount of Tau in the pancreas is elevated compared to normal cells.

Parkinson's: “Studies have shown that most people with PD have lost 60 to 80% or more of the dopamine-producing cells in the substantia nigra area of the brain by the time symptoms appear.”
National Institute of Neurological Disorders and Stroke (.gov) https://www.ninds.nih.gov>disorders>parkinsons-disease

“Feb. 25, 2024 New research identifies tau protein as a key player in the early stages of Parkinson's disease, challenging traditional views and suggesting new directions for treatment. . . . The findings also underscore the potential of targeting tau pathology as a therapeutic approach in Parkinson's disease. Because tau aggregation correlates with motor deficits and degeneration of dopamine-producing regions of the brain, interventions aimed at reducing tau accumulation could offer new hope for altering the disease's trajectory.
Unveiling the Shadow Player: Tau Protein's Silent Role in Parkinson's Disease
   scitechdaily.com>unveiling-the-shadow-player-tau Unveiling the Shadow Player: Tau Protein's Silent Role in . . . _Interview with Kordower: Brain. DOI: 10.1093/brain/awad388

“Of note, pancreatic tau is significantly up-regulated not only in T2DM but also in neurodegenerative diseases like . . . Parkinson's disease . . . .³
Martinez-Valbuena, I. et al. Mixed pathologies in pancreatic beta cells from subjects with neurodegenerative diseases and their interaction with prion protein. Acta Neuropathol. Commun. 9, 64 (2021).

### Diabetes Tau in Pancreas

Tau is known to multiply within the pancreas; however, there are 1 Billion pancreatic beta cells capable of producing the needed insulin. “Tau levels were found to be elevated in β-islet {pancreatic insulin producing} cells of patients with T2DM, . . . These findings present tau as a common therapeutic target between AD and T2DM.”
Tau suppresses microtubule-regulated pancreatic insulin secretion.
Mangiafico, S. P., Tuo, Q Z., Li, X L. et al. Mol Psychiatry 28, 3982-3993 (2023). https://doi.org/10.1038/s41380-023-02267-w
   AI SUMMARY: In Type 2 diabetes, significant {pancreatic} beta cell dysfunction occurs, often with 50-70% loss before diagnosis.
Type 1 diabetes does not present clinically until 80-90% of the beta cells have been destroyed (McCance & Heuther, 2014).
McCance, K. L. & Huether, S. E. (2014). Pathophysiology: The biological basis for disease in adults and children (7th ed.). St. Louis, MO: Mosby.
“In type 1 diabetes, β-cell mass is reduced by 70-80% at the time of diagnosis. Because of the variable degrees of insulitis and absence of detectable β-cell necrosis, it was suggested that β-cell loss occurs slowly over years (2).
“In type 2 diabetic subjects, initial pathological studies suggested a β-cell loss of 25-50% (2), (5)
Miriam Cnop, Nils Welsh, Jean-Christophe Jonas, Anne Jörns, Sigurd Lenzen, Decio L. Eizirik; Mechanisms of Pancreatic β-Cell Death in Type 1 and Type 2 Diabetes: Many Differences, Few Similarities. Diabetes 1 Dec. 2005; 54 (suppl_2): S97-S107. https://doi.org/10.2337/diabetes.54.suppl_2.S97

### Parkinson'S Tau in Brain

alpha-synuclein (α-syn) is an Amyloid oligomer (Aβ) associated with Parkinson's Disease

“Tau and α-syn can cross-seed each other in the process of aggregate formation and spreading.

The present review provides an update on disease classification of neurodegeneration and suggests niches of pathology intervention at early stages of tauopathies and synucleinopathies.”
Li, W., Li, J Y. Overlaps and divergences between tauopathies and synucleinopathies: a duet of neurodegeneration. Transl Neurodegener 13, 16 (2024). https://doi.org/10.1186/s40035-024-00407-y
Tau forms oligomeric complexes on microtubules that are distinct from tau aggregates, M. T. Gyparaki, A. Arab, E. M. Sorokina, A. N. Santiago-Ruiz, C. H. Bohrer, J. Xiao, M. Lakadamyali, Proc. Natl. Acad. Sci. U.S.A. 118 (19) e2021461118, 2021
Copyright © 2025 National Academy of Sciences. All rights reserved.|Online ISSN 1091-6490 PNAS is a partner of CHORUS, CLOCKSS, COPE, CrossRef, ORCID, and Research4Life.

### Another Benzimidazole: Mebendazole 1984

In 1984, 12 diabetic patients, for whom traditional treatments had failed, were treated with Mebendazole. “Included were six Type 1 (insulin-dependent) and six Type 2 (non-insulin-dependent) diabetic patients; eight of whom were chronically resistant to conventional treatment.”

Mebendazole treatment resulted in decreased plasma glucose concentration, (increased insulin secretion, and increased C-peptide).
Type 1 Diabetics 12.83 lowered to 6.56 mmol/l
Type 2 Diabetics 10.22 lowered to 7.56 mmol/l

Treatment had lasted for a short time, 1 month, using a low dose of 100 mg to 300 mg daily.

After patients were then taken off of Mebendazole:
metabolic control deteriorated in all the Type 2 diabetic patients
metabolic control deteriorated in five of the six Type 1 diabetic patients
In one of the six Type 1 diabetic patients, “the fasting plasma glucose concentration approached normality, and his insulin doses were gradually and persistently decreased throughout a 5-month period after discontinuation of Mebendazole. At that time insulin therapy was withdrawn and chlorpropamide therapy was started.”

Caprio and Owen, in a follow-up article, found that Mebendazole (testing a rat pancreatic islet) resulted in increased insulin secretion:
“To investigate the direct effect of Mebendazole on insulin secretion we used intact islets isolated from normal rat pancreata.
Mebendazole in concentrations as low as 10 to 20 mol/L caused a twofold to threefold increase in acute-phase insulin release from isolated perifused rat islets.

Caprio concluded: “However, these beneficial results may be transient.”

In a 1985 follow-up study, Van Gaal, using Mebendazole (supplied as Vermox from Janssen Pharmaceuticals, Beese, Belgium) treated 12 Type 1 diabetic patients for 30 days: “Although there was a trend towards a decrease in blood glucose levels at day 30, this decrease was not significant . . . our results do not confirm the findings of their preliminary study.”
Van Gaal L F, De Leeuw I H. Influence of mebendazole administration on metabolic control in type 1 (insulin-dependent) diabetic patients. Diabetologia. April; 28(4):250. 1985 doi: 10.1007/BF00282242. PMID: 4018451 No abstract available./Letters to Editor with DataTable https://link.springer.com/article/10.1007/BF00282242
Caprio, S. et al. Improvement of metabolic control in diabetic patients during mebendazole administration: preliminary studies. Diabetologia 27, 52-55 (1984).
Owen, O. E. et al. {CAPRIO is an author here} Mebendazole and insulin secretion from isolated rat islets. Metab. Clin. Exp. 34, 567-570 (1985).

### Chemical Structure Benzimidazole Family: Fenbendazole, Mebendazole

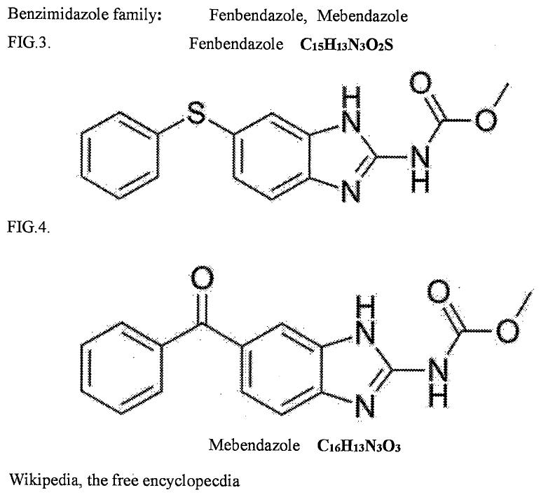

FIG. 3. Fenbendazole C₁₅H₁₃N₃O₂S

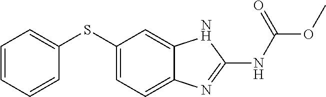 [SMILES: COC(=O)Nc1nc2ccc(Sc3ccccc3)cc2[nH]1]

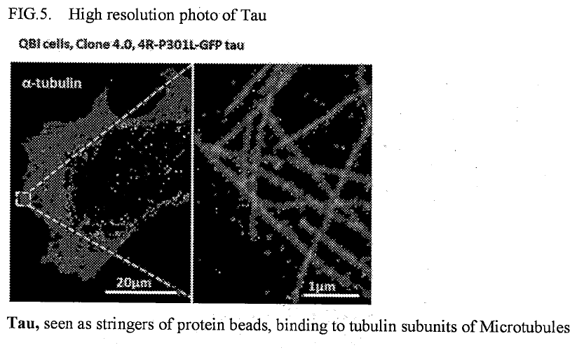

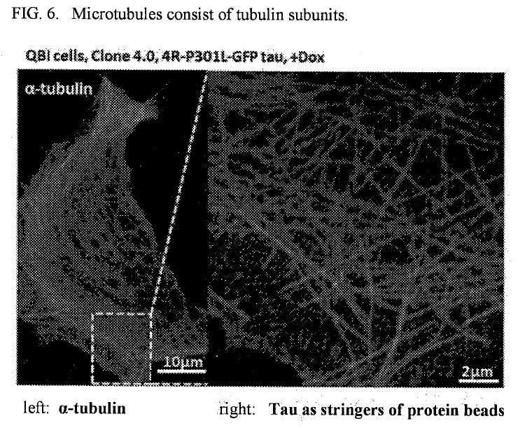

FIG. 4.

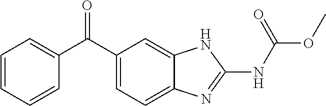 [SMILES: COC(=O)Nc1nc2ccc(C(=O)c3ccccc3)cc2[nH]1]

Mebendazole C₁₆H₁₃N₃O₃

Wikipedia, the free encyclopedia

### DESCRIPTION OF THE INVENTION

### Mechanism Disrupting Excess Tubulin/Microtubules

Fenbendazole disrupts excess intracellular tubulin and/or Microtubules, which selectively clears problematic cells.

### Technical Evaluation Report Fenbendazole

May 13, 2020 line 69, 122, 249, 709 [USDA] US Dept. of Agriculture Technical Report—Fenbendazole—May 13, 2020

For example: the Fenbendazole mechanism has previously cleared metastatic cancer; disrupting intracellular tubulin and/or Microtubules.
Sultana, T., Jan, U., Lee, H., Lee, H., & Lee, J. I. (2022). Exceptional Repositioning of Dog Dewormer: Fenbendazole Fever. Current Issues in Molecular Biology, 44(10), 4977-4986. https://doi.org/10.3390/cimb44100338

Similarly, researchers have been seeking a way to control Diabetes at the cellular level. Recent high resolution Microscopy reveals Tau attached to the Microtubules.

High resolution photo of Tau: Tau, seen as stringers of protein beads, binding to tubulin subunits of Microtubules.

Taken by Storm: The Rise and Fall of Tau from Microtubule-Associated to Aggregated to Degraded
page 86 C. Fig. A2
Melina Theoni Gyparaki, A DISSERTATION in Biology Presented to the Faculties of the University of Pennsylvania in Partial Fulfillment of the Requirements for the Degree of Doctor of Philosophy 2022 Supervisor of Dissertation Dr. Melike Lakadamyali Associate Professor of Physiology Graduate Group Chairperson Dr. Brian Gregory Professor of Biology

### Parkinson'S Dopamine Production in Brain Cells

For example, Tau oligomers have been found to seed and spread. Retinal manifestations of Tau and or Amyloid are a biomarker. From the eyes, Tau has been known to proliferate through nerve cells, reaching the brain.

“pTau pathology from the retina to the brain requires abundant pTau seeds, notably present in the AD brain”
Walkiewicz, Grzegorz & Ronisz, Alicja & Ospitalieri, Simona & Tome, Sandra & Vandenberghe, Rik & De Groef, Lies & Thal, Dietmar. (2025). Seeding and propagation of Tau from the retina to the brain in TAU58 mice. Alzheimer's & Dementia. 20. 10.1002/alz.087453. https://doi.org/10.1002/alz.087453 https://creativecommons.org/licenses/by/4.0/

“Several in vitro and in vivo studies have shown how α-syn, Aβ and tau can be transmitted via a “prion-like” mechanism in cellular and animal models (Soto, 2012; Goedert, 2015; Stopschinski and Diamond, 2017). These features may explain the high toxicity of α-syn oligomers and why they displayed such pronounced seeding properties (Fusco et al., 2017). More recently, it was shown that α-syn fibrils can release α-syn oligomers in vitro (Cascella et al., 2021).
Zampar S, Di Gregorio S E, Grimmer G, Watts J C and Ingelsson M (2024) “Prion-like” seeding and propagation of oligomeric protein assemblies in neurodegenerative disorders. Front. Neurosci. 18:1436262. doi: 10.3389/fnins.2024.1436262

“ . . . The findings also underscore the potential of targeting tau pathology as a therapeutic approach in Parkinson's disease. Because tau aggregation correlates with motor deficits and degeneration of dopamine-producing regions of the brain, interventions aimed at reducing tau accumulation could offer new hope for altering the disease's trajectory.
“Nigrostriatal tau pathology in parkinsonism and Parkinson's disease” by Yaping Chu, Warren D Hirst, Howard J Federoff, Ashley S Harms, A Jon Stoessl and Jeffrey H Kordower, 25 Nov. 2023, Brain. DOI: 10.1093/brain/awad388

The Fenbendazole Mechanism clears excess Tau:{Tau, amyloid, and α-syn) by disrupting the tubulin/Microtubule transport of glucose, disrupting glucose uptake, and clearing toxic oligomers within individual cells. High levels of Tau precede brain pathology and promote neuronal dysfunction.
Chiasseu M, Alarcon-Martinez L, Belforte N, Quintero H, Dotigny F, Destroismaisons L, Vande Velde C, Panayi F, Louis C, Di Polo A. Tau accumulation in the retina promotes early neuronal dysfunction and precedes brain pathology in a mouse model of Alzheimer's disease. Mol Neurodegener. 2017 Aug. 3; 12(1):58. doi: 10.1186/s13024-017-0199-3. PMID: 28774322; PMCID: PMC5543446.

“The pathological hallmarks of Parkinson's disease (PD) are the progressive loss of 320 dopaminergic neurons in the substantia nigra and the formation of Lewy bodies (LBs) in remaining neurons . . . . However, accumulating evidence suggests that Tau, which is associated with tauopathies . . . is also involved in the pathophysiology of PD . . . . Tau interacts with α-Syn and influences the pathology of α-Syn in PD. In this review, we discuss the structure and function of Tau and provide a summary of the current evidence supporting Tau's involvement as either an active or passive element in the pathophysiology of PD, which may provide novel targets for the early diagnosis and treatment of PD.
Keywords: Parkinson's disease, α-Synuclein, Tau, Pathophysiology
Pan L, Meng L, He M, Zhang Z. Tau in the Pathophysiology of Parkinson's Disease. J Mol Neurosci. 2021 November; 71(11):2179-2191. doi: 10.1007/s12031-020-01776-5. Epub 2021 Jan. 18. PMID: 33459970; PMCID: PMC8585831.
Ho W L, Leung Y, Tsang A W, So K F, Chiu K, Chang R C. Review: tauopathy in the retina and optic nerve: does it shadow pathological changes in the brain? Mol Vis. 2012; 18:2700-10. Epub 2012 Nov. 12. PMID: 23170062; PMCID: PMC3501278.

### Diabetes

### 1) Insulin Production Pancreatic Cells

For example, high resolution microscopy has found Tau attached to pancreatic diabetic cells. In 345 the pancreas, there are originally a billion beta cells (“islet cells”). By the time that T2DM has been diagnosed, at least 25-50% of pancreatic insulin producing cells have died . . . and the insulin production is reduced.

### 2) Hyperglycemia

As the Tau infestation advances, the number of Microtubules and the length of Microtubules increase steadily. GLUT4 glucose transporters struggle to pass amongst the density of hundreds to thousands of Microtubules (inside a single cell). GLUT4 glucose pickup and delivery from the cell membrane is slowed.
Both Diabetes and Parkinson's diseases become Hyperglycemic (high glucose in blood vessels).
Both Diabetes and Parkinson's use GLUT4 glucose transport with Microtubules.

Meanwhile, Normal cells begin to store the High Blood glucose as fats, resulting in Obesity
Hyperglycemia-driven signaling bridges between diabetes and . . .
Nov. 1, 2024 ⋅ Growing epidemiological evidence indicates an association between obesity, type 2 diabetes, and certain cancers, suggesting the existence of common underlying mechanisms . . .

### 3) Increased Risk of Stroke

Problematic Tau cells are still hungry long after meals. Their long slow steady intake of glucose competes with normal cells, and can deprive and starve the heart muscles of glucose. “A systematic review . . . identified hypoglycemia, {low blood sugar} . . . as contributing to cardiovascular risk in diabetic patients¹⁰
Exposure to hypoglycemia and risk of stroke. Ann N Y Acad Sci. 2018 November; 1431(1):25-34. doi: 10.1111/nyas.13872. Epub 2018 Jun. 19. PMID: 29917249; PMCID: PMC6214717. Smith L, Chakraborty D, Bhattacharya P, Sarmah D, Koch S, Dave K R.

“Individuals with T2DM had a significantly higher resting-energy expenditure than the healthy cohort “
https://discoveries.vanderbilthealth.com/2025/01/study-challenges-assumptions-in-type-2-diabetes/
Shen, Xinai, Huan Li, Beiyu Zhang, Yunan Li, and Zheying Zhu. 2025. “Tau-Targeted Therapeutic Strategies: Mechanistic Targets, Clinical Pipelines, and Analysis of Failures” Cells 14, no. 19: 1506. https://doi.org/10.3390/cells14191506

### 4) High Microtubule Density

Microtubules—Jones & Bartlett Learning
https://samples.iblearning.com/0763739057/lewincellbio_ch07.pdf

“Interphase cells {prepares for division} often contain hundreds of long microtubules that run throughout the cyto-plasm and connect one area of a cell to another, {intracellular}

A typical eukaryotic cell can contain thousands of microtubules, depending on its size and type. In larger cells, such as neurons, the number of microtubules can be significantly higher.

Microtubules consist of tubulin subunits.
Tau forms oligomeric complexes on microtubules that are distinct from tau aggregates, M. T. Gyparaki, A. Arab, E. M. Sorokina, A. N. Santiago-Ruiz, C. H. Bohrer, J. Xiao, M. Lakadamyali, Proc. Natl. Acad. Sci. U.S.A. 118 (19) e2021461118, 2021 (FIG. S1.C) Copyright © 2025 National Academy of Sciences. All rights reserved.|Online ISSN 1091-6490 PNAS is a partner of CHORUS, CLOCKSS, COPE, CrossRef, ORCID, and Research4Life.

### 5) Increased Tau

### Tau Stabilizes/Increases Microtubules

Tau preferentially moves closer to the cell membrane.

The seeding and proliferation of Tau increases.

The proliferation of Tau utilizes the High Blood glucose

For example: in cancer cells, proliferation is aided by high Microtubule density.

“Trogden notes that during diabetes, microtubules are much denser inside beta cells.”

“Trogden and other researchers in the Kaverina laboratory are now looking into ways to help stabilize microtubule density in beta cells during type 2 diabetes.”
https://discoveries.vanderbilthealth.com/2019/09/glucose-triggers-more-microtubules-and-insulin-release/

### 6) Normalizing Glucose Levels

Both Diabetes and Parkinson's and many other neurodegenerative diseases are correlated to increasing Hyperglycemia. Fortunately, Mebendazole and Fenbendazole have been successfully clearing Hyperglycemia for 50 years.

### Patents “Fenbendazole AND Diabetes”

### Research Documents

Parasites

1: A method for the treatment or prevention of Type 2 Diabetes in a subject in need thereof, comprising administering to the subject a therapeutically effective amount of Fenbendazole or a pharmaceutical composition comprising Fenbendazole, wherein said administration modulates microtubule vesicle trafficking to reduce pathological microtubule density inside beta cells and improve glucose homeostasis.

2: A method for the treatment or prevention of Parkinson's disease in a subject in need thereof, comprising administering to the subject a therapeutically effective amount of Fenbendazole or a pharmaceutical composition comprising Fenbendazole, wherein said administration modulates microtubule polymerization to inhibit hyperphosphorylated tau oligomerization and neurodegeneration.

3: A method for the treatment or prevention of Type 2 Diabetes in a subject in need thereof, comprising administering to the subject a therapeutically effective amount of Mebendazole or a pharmaceutical composition comprising Mebendazole, wherein said administration modulates microtubule vesicle trafficking to reduce pathological microtubule density inside beta cells and improve glucose homeostasis.

4: A method for the treatment or prevention of Parkinson's disease in a subject in need thereof, comprising administering to the subject a therapeutically effective amount of Mebendazole or a pharmaceutical composition comprising Mebendazole, wherein said administration modulates microtubule polymerization to inhibit hyperphosphorylated tau oligomerization and neurodegeneration.

5: The method of claims 1, 2, 3, or 4, wherein the composition is formulated for oral administration, parenteral administration, or rectal administration, and is made up in a physical form selected from the group consisting of solid forms, capsules, tablets, pills, granules, powders, suppositories, liquid forms, solutions, suspensions, emulsions, paste forms, nasal forms, buccal forms, sublingual forms, topical forms, inhalational forms, transdermal forms, subcutaneous injections, intravenous injections, intrahepatic injections, intramuscular injections, aqueous solutions, ointments, creams, gels, sprayable forms, aerosol forms, and nanoparticle formulations, together with one or more pharmaceutically acceptable carriers or excipients.

6: The method of claim 5, wherein the pharmaceutical composition further comprises an additional component selected from the group consisting of berberine, curcumin, quercetin, eugenol, rosmarinic acid, kaempferol, and combinations thereof, wherein the composition optionally includes Carbon 60 (C60) as an absorption carrier.

7: The method of claim 5, wherein the active ingredient is formulated to deliver a daily target baseline dosage ratio of approximately 200 mg per 200 pounds of subject body weight, administered with food, wherein the dosage is titrated or temporarily discontinued based on the subject's biological metrics, tolerance, and adverse side effects.
